(19) United States
(12) Patent Application Publication
(10) Pub. No.: US 2013/0046200 A1
(43) Pub. Date: Feb. 21, 2013

(54) Instrument For Concurrent Injection Of Anesthesia And Removal Of Specimens From A Body

(71) Applicant: Marshall Ephraim Stauber, Hollywood, FL (US)

(72) Inventor: Marshall Ephraim Stauber, Hollywood, FL (US)

(21) Appl. No.: 13/212,863

(22) Filed: Aug. 18, 2011

(51) Int. Cl. A61B 10/02

(52) U.S. Cl. 600/567

(57) ABSTRACT

The instant invention describes a biopsy instrument which is useful in removal of tissue or fluid samples from a body, such as bone and soft tissue biopsy procedures. The biopsy instrument is an improvement over other biopsy needles currently in use in that the biopsy instrument according to the instant invention can be used to reduce the pain associated with specimen removal procedures. The biopsy instrument generally comprises an outer cannula and an inner medical probe member. The inner medical probe member is constructed and arranged to contain one or more fluid dispensing members for dispensing a fluid which are in fluid communication with a fluid dispensing device. In this arrangement, the user has the ability to continually inject fluids, such as anesthetizing agents, directly at or near the spot of specimen removal, without the need to remove the surgical instrument or use a separate fluid injecting needle.

### FIELD OF THE INVENTION

This invention relates generally to surgical equipment, and in particular, surgical equipment that reduces pain and discomfort to a patient undergoing a biopsy procedure; and more particularly, a biopsy needle with the capability of dispensing anesthetizing agents at or near the site of biopsy as well as at or near the time of insertion of the biopsy needle into a specimen, such as soft tissue or bone, in order to remove and analyze such specimen.

### BACKGROUND OF THE INVENTION

Some diseases and illnesses can be diagnosed using relatively non-intrusive procedures such as blood tests or nasal or throat culturing. Other diseases, such as most cancers, require more intrusive diagnostic procedures. Once such procedure, the biopsy, is generally performed to confirm the diagnosis of a particular disorder that was found on another test or to differentiate between benign and malignant masses. Biopsy procedures require the removal of a patient's tissue or cellular fluid in order to provide a more accurate diagnosis and possible treatment modality. During the procedure, a tissue specimen can be removed with the aid of a needle. The needle is inserted through the skin and directly into the specimen. Alternatively, tissue samples can be removed through direct cutting and removal of the tissue.

Most biopsy procedures using biopsy needles require the patient to receive an injection of local anesthesia prior to retrieval of the sample. The surgeon injects the area where the biopsy needle is to be inserted, resulting in the numbing of a small, localized area. The result of the injection of the anesthesia, however, is not always complete. Failure to completely numb the area results in pain or discomfort during the process. For example, the patient often feels a sharp pain as fluid specimen samples are removed. Insertion of the biopsy needle into a tissue often results in a brief, usually dull pain. Moreover, patients undergoing bone biopsy commonly report a sharp pain as the needle enters into the bone. As the bone sample is removed, patients commonly complain of an additional aching pain or pressure. Such problems associated with biopsy procedures can be attributed to the anesthesia not reaching the deep tissue regions where the biopsy occurs or as a result of the chemicals rapidly diffusing to the surrounding area.

While the surgeon can inject the numbing agents into a general area or use multiple injections, the ability to direct the agent to the desired location can be problematic. Multiple injections subject the patient to pain, without alleviating the underlying problem. Moreover, most biopsy needles are cumbersome and difficult to work with, and obtaining a sample often requires multiple attempts. In some instances, the surgeon is required to move the biopsy needle from the original spot of entry to effectively obtain the sample. If the area around the biopsy needle is not fully anesthetized, the patient may suffer increased pain and discomfort. Should the surgeon need to inject more anesthetizing agents, current procedure would require insertion of a second needle, thereby increasing the patient's discomfort and pain levels associated with the procedure.

Various bone biopsy devices, such as those described in U.S. Pat. Nos. 4,266,555, 4,735,215, 4,838,282, and 5,425,376 have been used by physicians in performing biopsy procedures. Despite much effort to eliminate problems associated with biopsy procedures, physicians have not been able to develop a procedure that totally eliminates pain and the uncomfortable feeling patients often associate with these procedures. The shortcomings of biopsy procedures result from an inability to easily direct the anesthesia to a location that is at or near the site of the biopsy, particularly as the biopsy needle is inserted into the specimen. What is needed in the art, therefore, is a biopsy device that can inject fluids, such as anesthesia, at or near the site of the biopsy concurrently, or nearly concurrently, with the insertion of the needle into the specimen.

### SUMMARY OF THE INVENTION

The instant invention describes a surgical instrument which is useful in biopsy procedures, particularly bone or soft tissue biopsy procedures. The surgical instrument in accordance with the instant invention is an improvement over other biopsy needles currently in use and overcomes the shortcoming of the prior art by providing a device that reduces the pain associated with tissue specimen removal procedures. The biopsy instrument comprises a handle adapted for gripping with a hand, an outer cannula having a proximal end coupled to the handle, a distal end, and a main body therebetween. The main body has an inner lumen which is sized and shaped to co-axially align with a specimen removal device. The specimen removal device comprises a proximal end, a distal end, and a main body therebetween. The main body of the specimen removal device is sized and shaped to slidably engage with the inner lumen of the outer cannula. The biopsy instrument further comprises at least one fluid dispensing member for dispensing a fluid which is in fluid communication with a fluid dispensing unit. A method of concurrent injection of a fluid and removal of a specimen from a body is also described.

Accordingly, it is an objective of the instant invention to provide an improved surgical instrument which minimizes the pain and discomfort associated with biopsy procedures.

It is a further objective of the instant invention to provide an improved biopsy instrument which minimizes the pain and discomfort associated with tissue biopsy procedures.

It is yet another objective of the instant invention to provide an improved biopsy instrument which minimizes the pain and discomfort associated with bone biopsy procedures.

It is a still further objective of the invention to provide an improved biopsy instrument which is constructed and arranged to inject anesthetizing agents at or near the site of biopsy.

It is a further objective of the instant invention to provide an improved biopsy instrument which is constructed and arranged to inject anesthetizing agents at or near the site of biopsy during insertion of the needle into a soft tissue.

It is yet another objective of the instant invention to provide an improved biopsy instrument which is constructed and arranged to inject anesthetizing agents at or near the site of biopsy prior to insertion of the needle directly into bone.

It is a still further objective of the invention to provide an improved biopsy instrument which is constructed and arranged to inject anesthetizing agents at or near the site of biopsy without the need to reposition the needle during the procedure.

It is a further objective of the instant invention to provide a method for concurrent injection of a fluid and removal of a specimen from a body.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A is a perspective view of an illustrative embodiment of the biopsy instrument in accordance with the instant invention;

FIG. 1B is an exploded view of the biopsy instrument;

FIG. 2A is a perspective view of an illustrative embodiment of the biopsy instrument in accordance with the instant invention, illustrating the specimen removal member extended from the outer cannula;

FIG. 2B is a perspective view of an alternative embodiment of the biopsy instrument, illustrating a self-contained dispensing unit disposed of within a handle;

FIG. 3A is a partial longitudinal section of the biopsy instrument in accordance with the instant invention;

FIG. 3B is a partial longitudinal section of an illustrative embodiment of the biopsy instrument in accordance with the instant invention, illustrating multiple fluid dispensing members;

FIG. 3C is a partial longitudinal section of an illustrative embodiment of the biopsy instrument in accordance with the instant invention, illustrating an alternative embodiment of the fluid dispensing member;

FIG. 4A is an alternative embodiment of the biopsy instrument in accordance with the instant invention, illustrating the fluid dispensing member positioned within the tip;

FIG. 4B is a cross sectional view of the biopsy instrument illustrated in FIG. 4A;

FIG. 5A is an alternative embodiment of the biopsy instrument in accordance with the instant invention, illustrating the fluid dispensing member positioned along the outer surface of the medical probe member;

FIG. 5B is a cross sectional view of the biopsy instrument illustrated in FIG. 5A;

FIG. 6A is a partial longitudinal sectional view of the biopsy instrument illustrated in FIG. 4A;

FIG. 6B is a partial longitudinal sectional view of the biopsy instrument, illustrating a plurality of fluid dispensing members;

FIG. 6C is a perspective view of an illustrative example of the medical probe member containing a fluid dispensing member aperture within each face of the bi-beveled tip;

FIG. 6D is a perspective view of an illustrative example of the medical probe member containing a plurality of apertures within each face of the tri-beveled tip;

FIG. 7 is a schematic of the use of the biopsy instrument in accordance with the instant invention, illustrating the pre-insertion of the biopsy instrument into a patient;

FIG. 8 illustrates the biopsy instrument in accordance with the instant invention inserted into the patient;

FIG. 9 illustrates the biopsy instrument in accordance with the instant invention prior to insertion within the target bone;

FIG. 10 illustrates the biopsy instrument in accordance with the instant invention inserted within the target bone;

FIG. 11 illustrates the biopsy instrument in accordance with the instant invention inserted deep within the target bone;

FIG. 12 illustrates the biopsy instrument in accordance with the instant invention in which a sample of the bone has been inserted within the outer cannula and ready for removal;

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

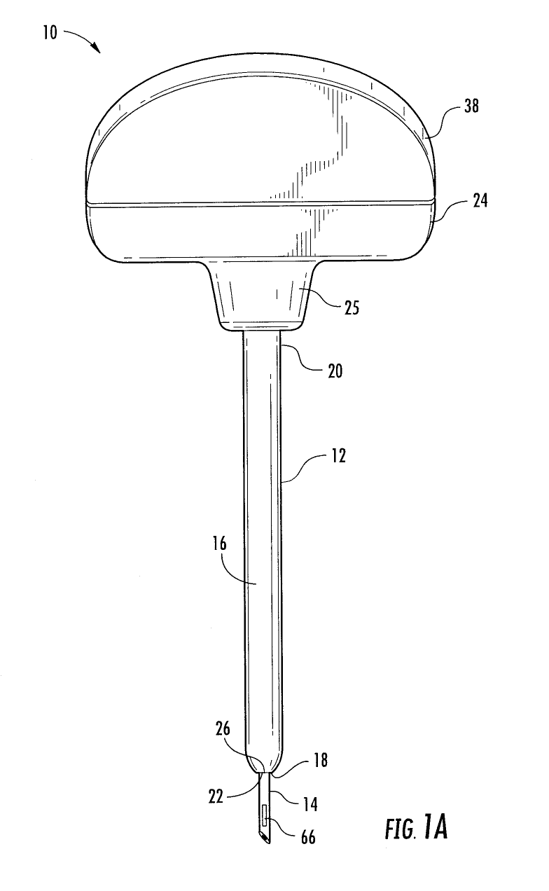

The instant invention describes a surgical instrument which is useful in bone and soft tissue biopsy procedures. The surgical instrument is an improvement over other biopsy needles currently in use in that the surgical device according to the instant invention overcomes the shortcoming of the prior art by providing a device that reduces the pain associated with tissue specimen removal procedures. Referring to FIGS. 1-6, a surgical instrument for concurrent injection of anesthetizing agents and removal of components of the body according to an embodiment of the present invention, herein referred to as a biopsy device, is indicated generally at 10. The biopsy device 10 may be, for example, a Jamshidi type device. The biopsy device 10 is preferably made of stainless steel, but can be made of other materials commonly used in surgical procedures. While the biopsy device 10 is primarily useful for removal of bone tissue or soft tissue in a human, other body samples may be removed using such device. Moreover, while the preferred use is for humans, such device may be useful in obtaining tissue or other internal body samples of other mammalians as well.

Referring to FIGS. 1-3, the biopsy device 10 comprises a specimen removal member, illustrated herein as an outer cannula 12, and an inner medical probe member 14. The inner medical probe member 14 is illustrated herein as a stylet. Other devices that remove specimens, such as a needle, or act as a probe, such as a trocar, known to one of skill in the art and useful in biopsy procedures can be used as well. The outer cannula 12 is preferably rod shaped having an outer surface 16 which defines the outer surface of the biopsy instrument 10 and a generally hollow interior 18. The outer cannula 12 is constructed and arranged to slidably engage the stylet 14 in a generally co-axial alignment. The outer cannula 12 contains a proximal end 20 and a distal end 22. A handle 24 attaches to the proximal end 20 via any means known to one of skill in the art, such as, but not limited to, threading or chemical fastening. A cap 25 may be used to cover the junction between the handle 24 and proximal end 20. The handle 24 may provide a gripping means for the user.

The distal end 22 of the outer cannula 12 contains an aperture 26 which is sized and shaped to provide the stylet 14 the ability to slide in and out of the hollow interior 18. The distal end 22 may include a tapering 28 so as to slidably engage the stylet 14 in a sealable arrangement to prevent portions of the specimens, such as fluids or tissue, from entering into the hollow interior 18. Although not illustrated, the distal end 22 may contain sharp, pointed edges or teeth-like structures to aid in insertion of the instrument into a specimen and/or the removal of a portion of such specimen. Alternatively, the distal end may contain a Franssen style, triple crown cannula tip, or a Wescot style tip.

The stylet 14 is sized and shaped to slidably fit within the outer cannula 12. It comprises a generally rod shaped body 30 having a proximal end 32 attached to a gripping/insertion handle 38 and a distal end 40. Referring to FIG. 1B, the distal end 40 contains a sloped surface 42, or face, which extends downwardly from the first surface 44 of the rod shaped body 30 to a second surface 46 of the rod shaped body 30. A sharp tip 48 aids the user when inserting the biopsy device 10 into a person and/or their internal tissue, organ, or bone. The distal end 40 of the stylet 14 need not contain a pointed end and may optionally have, for example, a blunt or rounded tip. The generally rod shaped body 30 of the stylet 14 is sized and shaped to slidably engage within the hollow interior 18 of the outer cannula 12. The stylet 14 can be inserted within the cannula 12 through an opening 50 in the handle 24. The stylet handle 38 may be secured to the handle 24 through use of securing members, such as a tab and tab receiving area (not illustrated). Other means of securing the stylet to the outer cannula, such as those described in U.S. Pat. Nos. 5,807,275, 5,331,972, or U.S. Patent Application Publication 2004/0077973, the contents of which are herein incorporated by reference, may be used.

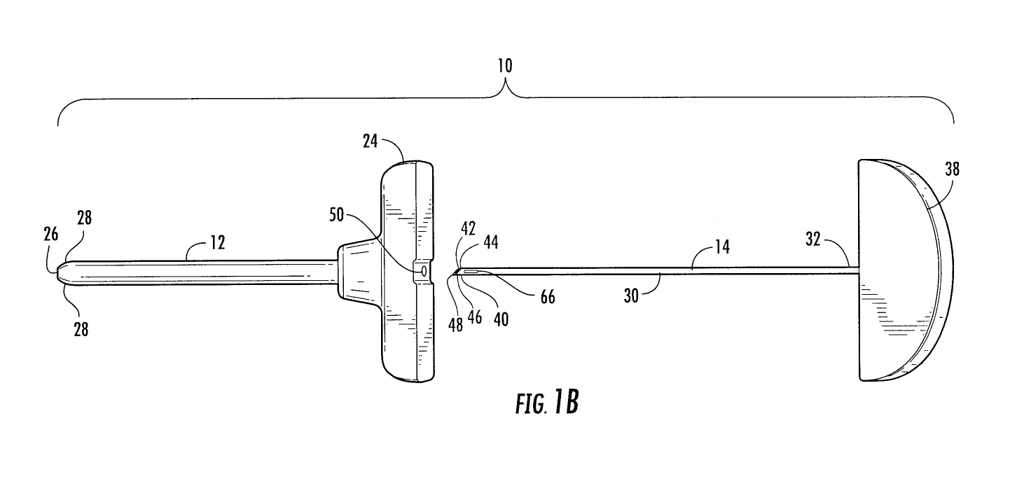

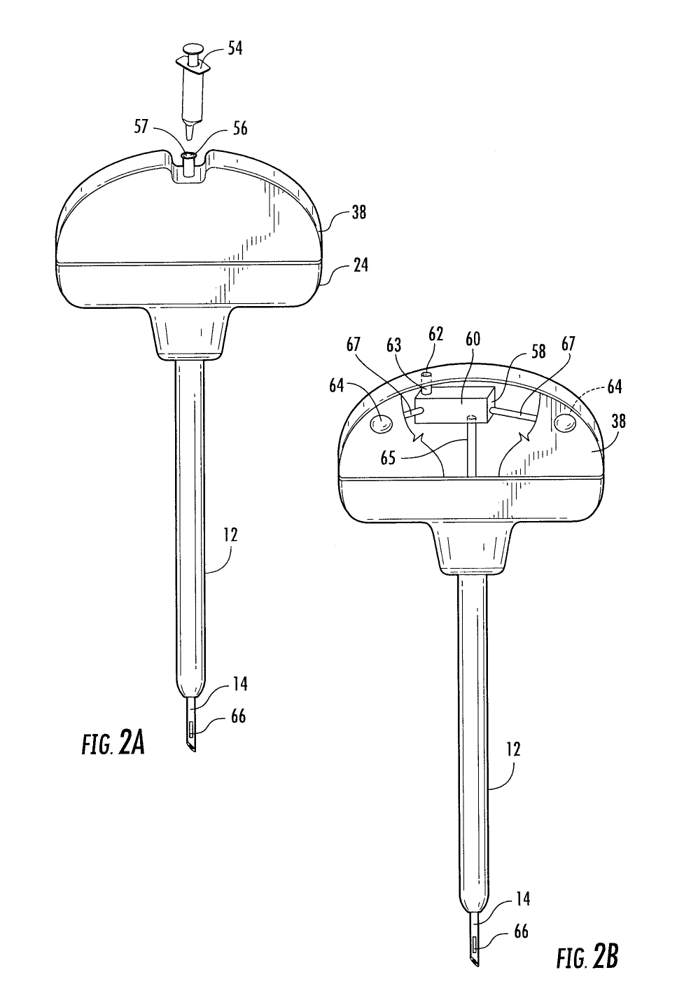

The stylet 14 further includes one fluid dispensing member 52, illustrated herein as an internal lumen, see for example FIG. 3A, for dispensing a fluid to a location, or a plurality of fluid dispensing members 52, see FIG. 3B. Alternatively, the stylet 14 may contain a single main fluid dispensing member 52 positioned through the center of the stylet and containing a plurality of branches 52′, 52″ as the dispensing member extends toward the distal end, see FIG. 3C. The internal lumen 52 can be sized to span the entire length of the outer cannula 12, or a portion thereof. The internal lumen 52 can be adapted to be in fluid communication with a fluid dispensing device 54, illustrated herein as a syringe, see FIG. 2A. The syringe 54 may be inserted into the biopsy device 10 through the handle 38 through, for example, a male/female Luer connector 56. The male/female Luer connector 56 contains an opening 57 to allow direct injection of a fluid within.

An alternative embodiment of the biopsy device 10 may include a self-contained fluid dispensing device 58, see FIG. 2B, placed within handle 38. The self-contained fluid dispensing device 58 may include a fluid storage unit or reservoir 60 which is in fluid communication with a port 62 through fluid passageway 63 and the one or more internal lumens 52 through fluid passageway 65. The port 62 may further contain a resealing membrane, or septum, to allow for multiple fillings of the fluid storage unit 60 with a fluid through the use of a needle attached to a syringe. One or more actuators, illustrated herein as fluid release buttons 64, in operable connection with the self-contained fluid dispensing device 58, are adapted to allow the user the ability to dispense and control the flow of fluid. The fluid release buttons 64 may be positioned anywhere within the handle 38. Preferably, the fluid release buttons 64 are not placed on the top surface so that that the user is free to use that surface to tap the instrument into place if needed. The one or more fluid release buttons 64, for example, may be designed so that depressing each button provides a force which pushes the fluid into the lumen 52. The fluid release buttons 64 may be a bulb-type pump similar to a lawn mower or weed-eating priming bulb. Engaging the bulb forces air through lumen 67 and pushing fluid out of the fluid storage unit 60. The release buttons may be operatively connected to a pump, such as a diaphragm pump disclosed in U.S. Patent Application Publication Number 2007/0086831, or a pressure membrane pump described in U.S. Pat. No. 5,918,995, in which pressure or suction effect is obtained in order to transfer the fluid out of the reservoir 60 and into the fluid dispensing member 52. Alternatively, pressing the buttons 64 may open a valve (not shown), such as a duckbill valve, as described in U.S. Pat. No. 5,993,180, that allows fluid to move in one direction under pressure, but not in another direction; or cause movement of a plunger (not shown) to push down on the fluid, allowing the fluid to flow into the lumen 52. Upon activation of the buttons 64, i.e. applying a force, fluid would flow, either continuously or in pre-measured amounts (i.e. 1 ml dose, 5 ml dose, 10 ml dose), from the fluid storage unit 60 to the one or more internal lumens 52. An internal lumen aperture 66 is located at or near the distal end 40 of the stylet 14 and may be positioned off-center along the main body 30. The internal lumen aperture 66 provides a location for any fluid that is dispensed from the syringe 54 or the self-contained fluid dispensing device 58 to exit the biopsy device 10.

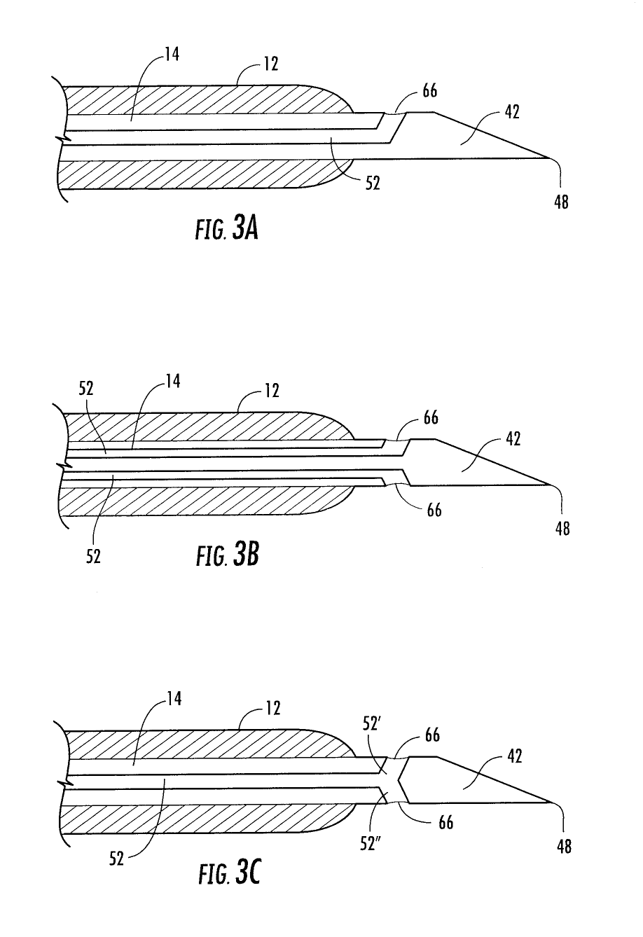

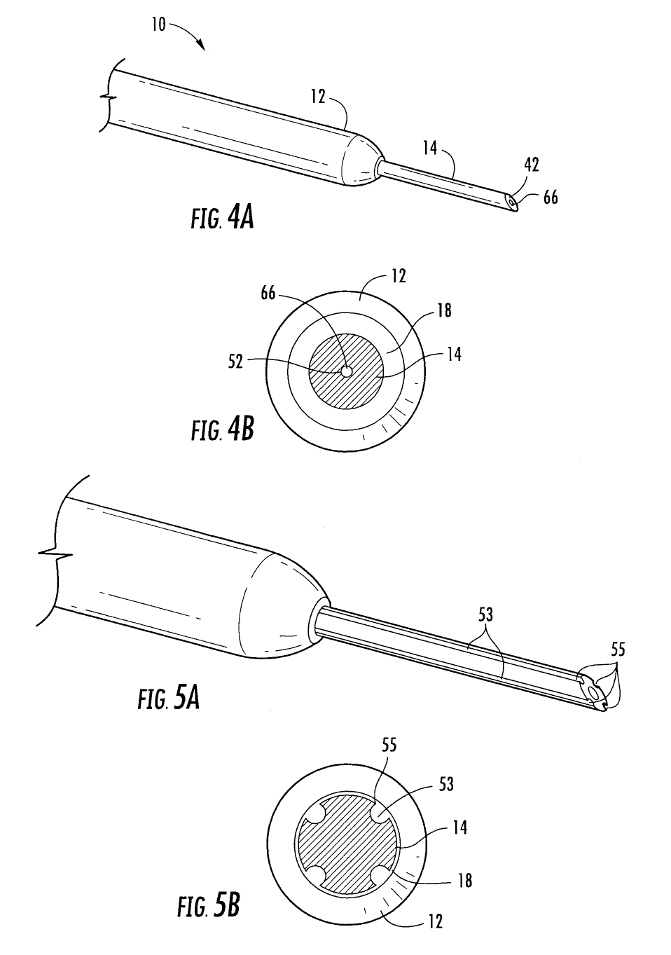

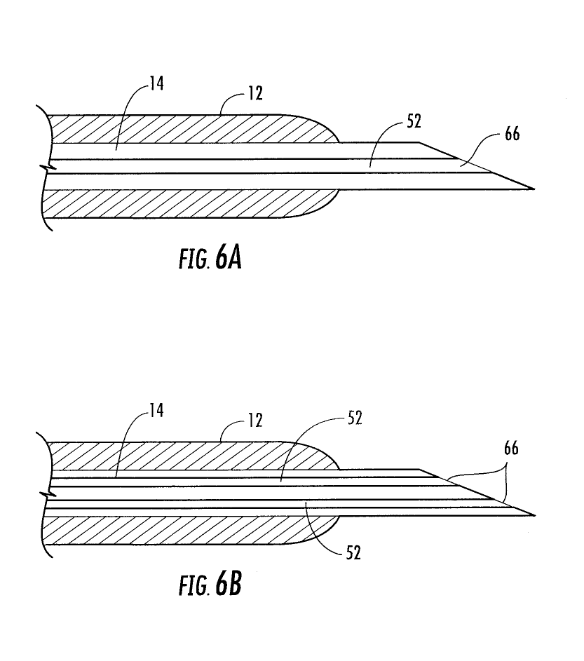

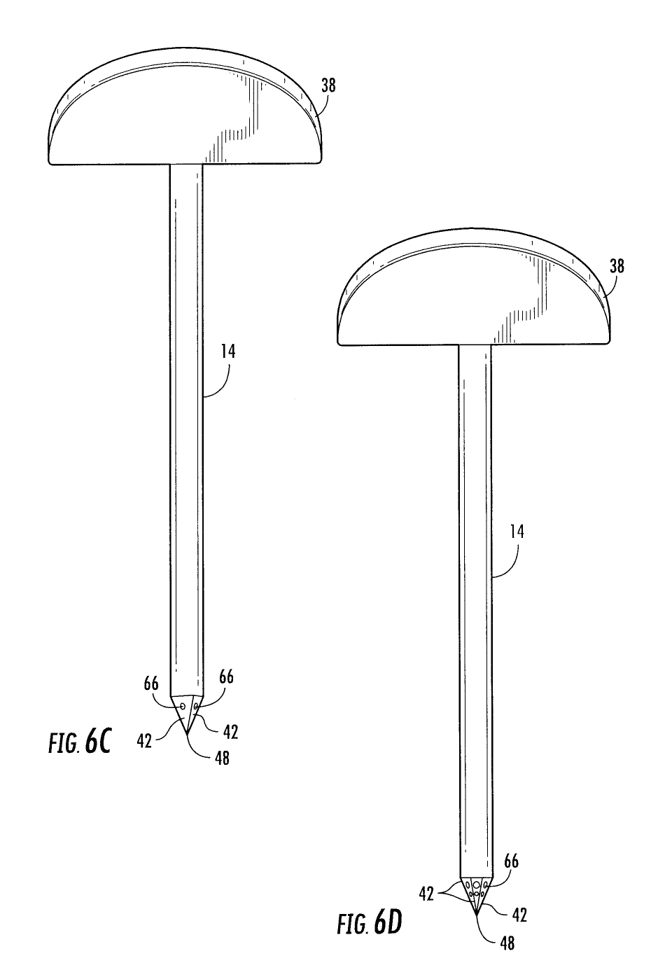

FIGS. 4A, 4B, and 6A illustrate an alternative embodiment of the biopsy device 10 in which the internal lumen aperture 66 is positioned on-center within the pointed tip of the stylet 14, within, for example, the sloped surface 42. FIG. 6B illustrates an alternative embodiment in which the sloped surface 42 contains a plurality of fluid dispensing members 52 and internal lumen apertures 66. FIGS. 6C and 6D illustrate the distal end of the stylet 14 in which the pointed end is multi-beveled. The bi-beveled tip shown in FIG. 6C contains at least one fluid dispensing member 52 terminating in aperture 66 in each of the faces of the two sloped surfaces 42. FIG. 6D illustrates a tri-beveled tip with a plurality of fluid dispensing members 52 in each of the faces three sloped surfaces 42. Having multiple fluid dispensing members 52 within the tip allows for radial dispensing of the fluid. FIGS. 5A and 5B illustrate an alternative embodiment of the biopsy device 10 in which the fluid dispensing members are formed as channels 53 running along the outer surface of the stylet 14. The channels terminate in half-moon shaped apertures 55.

The internal lumen 52 is preferably adapted for dispensing or injecting local anesthesia, such as lidocaine, or other materials, such as dyes or other visualizing materials, through soft tissue or directly into other specimens, such as bone, during insertion of the biopsy device 10. Injection of such numbing fluids at or near the site of biopsy provides an enhanced mechanism for minimizing pain during the procedure. In addition to the location of injection, the timing of the injection of the local anesthesia can be controlled, thereby further reducing pain associated with the biopsy and enhancing the biopsy experience for the patient. As opposed to traditional procedures which require the injection of numbing agents prior to insertion of the biopsy instrument site, the biopsy device 10 in accordance with the instant invention allows the user the ability to numb an area as the biopsy device is positioned into place. More importantly, since the local anesthetizing agent is localized to the specific area, the problems associated with applying the numbing agents through injection into the skin is minimized or reduced.

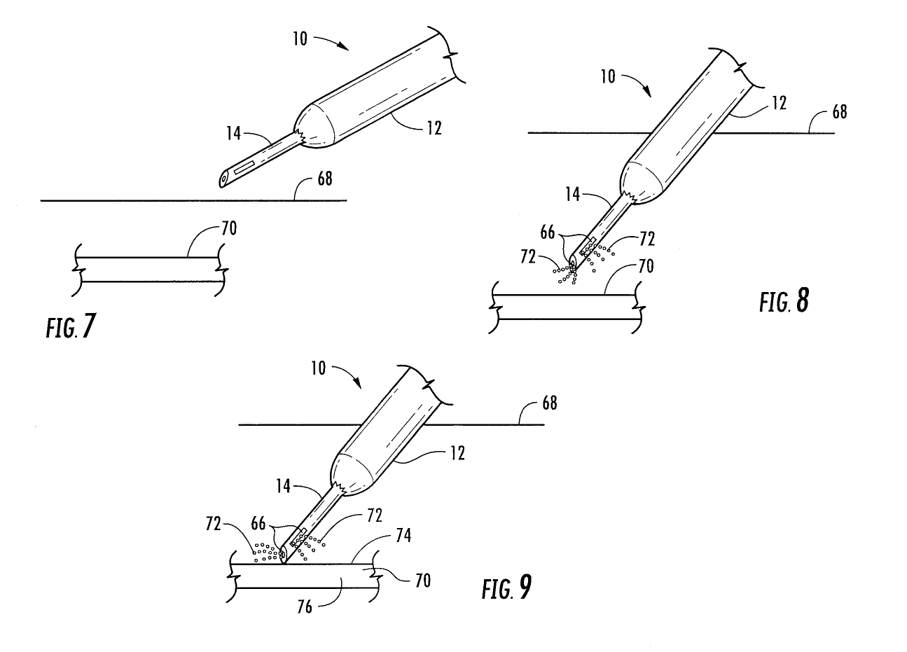

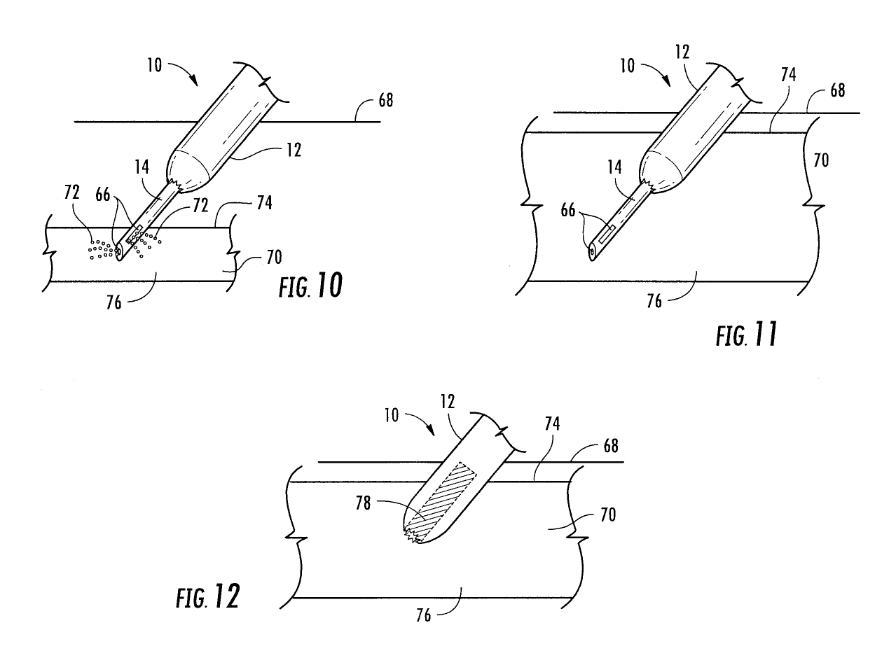

FIGS. 7-12 are schematic representations of the biopsy device 10 in use. FIG. 7 illustrates the biopsy device 10 just prior to insertion into the skin 68 of a patient in order to obtain a sample from a tissue, organ, or fluid 70 to be biopsied. As part of the general procedure, the patient has been injected with local anesthesia prior to insertion of the biopsy device 10. As illustrated in FIG. 8, as the biopsy device 10 is inserted into the patient, the user can dispense additional anesthesia agents 72 through the plurality of internal lumen apertures 66 to provide assurance that the direct area in which the biopsy device 10 contacts the patient is numbed, thereby reducing the amount of pain the patient may experience. As the biopsy device 10 is inserted deeper into the patient, the possibility that the initial injection of anesthesia remains is non effective and or has been dispersed to other parts of the body is high. The advantage of the instant invention is that as the user pushes the biopsy device further within the patient, the user has the ability to continually inject anesthetizing agents directly at or near the spot of biopsy contact, without the need to remove the biopsy device or use a second injecting needle. FIG. 9 illustrates the biopsy device prior to insertion into the periosteum 74 of the target, illustrated schematically as bone 70. Insertion of the needle into the periosteum 74 has been described by patients as a painful or an uncomfortable sensation. As the biopsy device 10 makes contact with the bone 70, it is possible that the area surrounding the bone 70, particularly the periosteum 74, may not be anesthetized or only partially anesthetized. Injecting additional anesthetizing agent 72 through the internal lumen apertures 66 provides local numbing and minimizes additional pain which may be suffered by the patient. The biopsy device 10 is inserted deeper into the core area 76 of the bone 70 in order to remove a sample, see FIG. 10. At the desired position, the stylet 14 is removed from the outer cannula 12, see FIG. 12. As the outer cannula 12 is then inserted deeper into the bone, a sample 78 is cored within the outer cannula 12, which can be removed for analysis.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An instrument for concurrent injection of a fluid and removal of a specimen from a body comprising:
a handle adapted for gripping with a hand;
an outer cannula having a proximal end coupled to said handle, a distal end, and a main body therebetween, said main body having an inner lumen sized and shaped to co-axially align with an inner medical probe member,
an inner medical probe member having a proximal end, a distal end, and a main body therebetween, said main body sized and shaped to slidably engage with said inner lumen of said outer cannula; and
at least one fluid dispensing member for dispensing a fluid.

2. The instrument for concurrent injection of a fluid and removal of a specimen from a body according to claim 1 wherein said at least one fluid dispensing member is a lumen traversing the length of said inner medical probe member.

3. The instrument for concurrent injection of a fluid and removal of a specimen from a body according to claim 2 wherein said at least one dispensing lumen is in fluid communication with a dispensing unit.

4. The instrument for concurrent injection of a fluid and removal of a specimen from a body according to claim 3 wherein said dispensing unit is a syringe.

5. The instrument for concurrent injection of a fluid and removal of a specimen from a body according to claim 3 wherein said at least one dispensing lumen is in fluid communication with a self-contained dispensing unit located in said handle of said inner medical probe member.

6. he instrument for concurrent injection of a fluid and removal of a specimen from a body according to claim 5 wherein said handle further comprises a port, said port in fluid communication with a fluid storage unit of said self-contained dispensing unit.

7. he instrument for concurrent injection of a fluid and removal of a specimen from a body according to claim 5 wherein said self-contained dispensing unit is adapted to continuously dispense fluid to at least one dispensing lumen.

8. The instrument for concurrent injection of a fluid and removal of a specimen from a body according to claim 5 wherein said self-contained dispensing unit is adapted to dispense fluid to at least one dispensing lumen in pre-determined amounts.

9. The instrument for concurrent injection of a fluid and removal of a specimen from a body according to claim 1 wherein said fluid is an anesthesia agent.

10. The instrument for concurrent injection of a fluid and removal of a specimen from a body according to claim 1 wherein said fluid is a visualizing material.

11. The instrument for concurrent injection of a fluid and removal of a specimen from a body according to claim 10 wherein said visualizing material is a dye.

12. The instrument for concurrent injection of a fluid and removal of a specimen from a body according to claim 2 wherein said lumen terminates in an aperture which is positioned off-center.

13. The instrument for concurrent injection of a fluid and removal of a specimen from a body according to claim 2 wherein said lumen terminates in an aperture, said aperture positioned within the tip of said inner medical probe member.

14. The instrument for concurrent injection of a fluid and removal of a specimen from a body according to claim 2 wherein said inner medical probe member contains a tip containing multiple surfaces, each of said surface fluidly connected to one or more said lumens and terminating in one or more apertures, said apertures adapted to dispense said fluid.

15. The instrument for concurrent injection of a fluid and removal of a specimen from a body according to claim 1 wherein said fluid dispensing member includes a plurality of channels running the length of said inner medical probe member, said channels fluidly coupled to a fluid dispensing unit.

16. The instrument for concurrent injection of a fluid and removal of a specimen from a body according to claim 15 wherein said plurality of channels run lengthwise along the outer surface.

17. The instrument for concurrent injection of a fluid and removal of a specimen from a body according to claim 1 wherein said inner medical probe member is a stylet.

18. A method of concurrent injection of a fluid and removal of a specimen from a body comprising the steps of:
determining the specimen required for removal from a body;
inserting a biopsy instrument into the patient, said biopsy instrument adapted for concurrent injection of a fluid and removal of a specimen from a body;
dispensing additional amounts of fluid from said biopsy instrument on an as needed basis, said dispensing of additional amounts of fluid from said biopsy instrument is performed prior to, or concurrently with, contact with said specimen, said fluid being dispensed directly at or near the spot of contact, wherein dispensing of additional fluid is accomplished without the need to remove the biopsy instrument from the patient or use a second injecting needle; and
removing said specimen from said body.

19. The method of concurrent injection of a fluid and removal of a specimen from a body according to claim 18 wherein said fluid is an anesthetizing agent, marking material, or combinations thereof.
